(19) United States
(12) Patent Application Publication
(10) Pub. No.: US 2023/0220437 A1
(43) Pub. Date: Jul. 13, 2023

(54) INCREASING YIELD OF STEVIOL GLYCOSIDES

(71) Applicant: CARGILL, INCORPORATED, Wayzata, MN (US)

(72) Inventors: Dan S. GASPARD, Victoria, MN (US); Adam T. ZARTH, St. Louis Park, MN (US)

(73) Assignee: CARGILL, INCORPORATED, Wayzata, MN (US)

(21) Appl. No.: 17/996,663

(22) PCT Filed: Apr. 20, 2021
(86) PCT No.: PCT/US2021/028162

§ 371 Date: Oct. 20, 2022

Related U.S. Application Data
(60) Provisional application No. 63/012,606, filed on Apr. 20, 2020.

(51) Int. Cl. C12P 19/56

(52) U.S. Cl. CPC C12P 19/56

(57) ABSTRACT

Various aspects of the present invention relate to steviol glycoside extraction. A method of treating a Stevia extract that includes steviol glycosides and/or salts thereof, and malonated steviol glycosides and/or salts thereof, includes treating the Stevia extract at a pH greater than 10 to convert at least some of the malonated steviol glycosides and/or salts thereof to non-malonated steviol glycosides and/or salts thereof, to produce a modified Stevia extract. The method includes decreasing the pH of the modified Stevia extract to a pH of less than 9 to provide a pH-adjusted modified Stevia extract. The method also includes recovering the steviol glycosides and/or salts thereof from the pH-adjusted modified Stevia extract, the recovered steviol glycosides and/or salts thereof including the non-malonated steviol glycosides and/or salts thereof.

### CROSS-REFERENCE TO RELATED APPLICATIONS

This application claims the benefit of U.S. Provisional Patent Application No. 63/012,606, filed Apr. 20, 2020, which is hereby incorporated by reference herein in its entirety.

### BACKGROUND

In recent decades, consumers have increasingly sought low-calorie alternatives to calorie-rich products. Steviol glycosides offer a non-caloric alternative to traditional caloric sweeteners such as sugar, glucose, sucrose, and/or fructose. Steviol glycosides are a class of sweet-tasting glycosylated diterpene compounds commonly obtained from the leaves of Stevia rebaudiana. Various steviol glycosides are known, some of which provide a sugar-like taste profile and are 150 to 450 times sweeter than sugar. Such compounds are typically characterized by a single steviol backbone and the presence of differing arrangements of glycosidic carbohydrate residues at positions C13 and C19.

### SUMMARY OF THE INVENTION

In various aspects, the present invention provides a method of treating Stevia extracts to convert malonated steviol glycosides and/or salts thereof to non-malonated steviol glycosides and/or salts thereof. Some implementations of the invention have been found to dramatically increase yield of steviol glycosides, which significantly improves the economics of steviol glycoside production.

In various aspects, the present invention provides a method of treating a Stevia extract that comprises steviol glycosides and/or salts thereof, and malonated steviol glycosides and/or salts thereof. The method includes treating the Stevia extract at a pH greater than 10 to convert at least some of the malonated steviol glycosides and/or salts thereof to non-malonated steviol glycosides and/or salts thereof, to produce a modified Stevia extract. The method includes decreasing the pH of the modified Stevia extract to a pH of less than 9 to provide a pH-adjusted modified Stevia extract. The method also includes recovering the steviol glycosides and/or salts thereof from the pH-adjusted modified Stevia extract, the recovered steviol glycosides and/or salts thereof comprising the non-malonated steviol glycosides and/or salts thereof.

In various aspects, the present invention provides a method of obtaining steviol glycosides from Stevia plant material. The method includes contacting the Stevia plant material with an aqueous extraction solution to form the Stevia extract. The method includes treating the Stevia extract at a pH greater than 10 to convert at least some of the malonated steviol glycosides and/or salts thereof to non-malonated steviol glycosides and/or salts thereof, to produce a modified Stevia extract. The method includes decreasing the pH of the modified Stevia extract to a pH of less than 9 to provide a pH-adjusted modified Stevia extract. The method also includes recovering the steviol glycosides and/or salts thereof from the pH-adjusted modified Stevia extract, the recovered steviol glycosides and/or salts thereof comprising the non-malonated steviol glycosides and/or salts thereof.

In various aspects, the present invention provides a method of obtaining steviol glycosides from Stevia plant material. The method includes contacting the Stevia plant material with an aqueous extraction solution at a pH of less than 9 to form a Stevia extract including steviol glycosides and/or salts thereof, and malonated steviol glycosides and/or salts thereof. The method includes treating the Stevia extract at a pH greater than 10 to convert at least some of the malonated steviol glycosides and/or salts thereof to non-malonated steviol glycosides and/or salts thereof, to produce a modified Stevia extract. The method includes decreasing the pH of the modified Stevia extract to a pH of less than 9 to provide a pH-adjusted modified Stevia extract; recovering the steviol glycosides and/or salts thereof from the pH-adjusted modified Stevia extract, the recovered steviol glycosides and/or salts thereof including the non-malonated steviol glycosides and/or salts thereof.

In various aspects, the present invention provides a method of increasing yield of steviol glycosides and/or salts thereof from a Stevia plant material. The method includes contacting the Stevia plant material with an aqueous extraction solution having a pH of 4 to 9, to form a Stevia extract. The method includes adding base to the Stevia extract to raise pH of the Stevia extract to 12 to 13. The method includes allowing the base to react with the Stevia extract at 15° C. to 30° C. for 1 minute to 60 minutes to form a modified Stevia extract. The method includes lowering the pH of the modified Stevia extract to 5 to 7. The method includes recovering steviol glycosides, salts thereof, or a combination thereof from the modified Stevia extract, to form a composition including the steviol glycosides, salts thereof, or a combination thereof. The steviol glycosides, salts thereof, or combination thereof, in the composition are obtained at a yield that is 1% to 250% greater than obtained from the same method without the adding of the base, the allowing of the base and the Stevia extract to react, and the lowering of pH.

Various aspects of the present invention provide advantages over other methods of extracting steviol glycosides and/or salts thereof from a Stevia plant material. For example, in various aspects, the present method can provide an increased yield of steviol glycosides and/or salts thereof from Stevia leaf, as compared to other methods that fail to hydrolyze malonated steviol glycosides and/or salts thereof to un-malonated steviol glycosides and/or salts thereof. In various aspects, the present method can include lowering the pH of the modified Stevia extract to neutral or near-neutral conditions after hydrolyzing malonated steviol glycosides, which can reduce equipment degradation, improve resin efficiency, decrease degradation of desired products, and enhance operator safety during recovery or purification of the steviol glycosides from the modified Stevia extract For example, by avoiding recovering or purifying steviol glycosides from a caustic mixture, damage to resin used for chromatographic treatment can be decreased or eliminated, and resin efficiency and useful lifespan can correspondingly increase. For example, by avoiding recovering or purifying steviol glycosides from a caustic mixture, deprotonation and ionization of sugar moieties on the steviol glycosides can be avoided, which can help to avoid yield loss during a hydrophobic adsorption step (e.g., product not sticking to resin) and during an ion exchange step (e.g., product sticking to anionic resin), thereby increasing overall yield of steviol glycosides.

### BRIEF DESCRIPTION OF THE FIGURES

The drawings illustrate generally, by way of example, but not by way of limitation, various embodiments of the present invention.

FIG. 1 illustrates a high resolution mass spectrometry (HRMS) spectrum of a Stevia leaf extract, with the circled peaks corresponding to malonate-containing compounds, in accordance with various aspects.

FIG. 2 illustrates an HRMS spectrum of a Stevia leaf extract that has been subjected to basic hydrolysis at pH 12.5, illustrating hydrolysis of malonate-containing compounds, in accordance with various aspects.

FIG. 3 illustrates response versus time for hydrolysis of Stevia leaf extract under various pH conditions at ambient temperature, in accordance with various aspects.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

Method of Extraction of Steviol Glycosides from Stevia Plant Material.

In various aspects, the present invention provides a method of extraction of steviol glycosides from Stevia plant material. The method can include extracting Stevia plant material, raising the pH to a suitable level to convert at least some malonated steviol glycosides to non-malonated steviol glycosides and/or salts thereof, lowering the pH, and then carrying out conventional steviol glycoside recovery.

The method can include contacting the Stevia plant material with an aqueous extraction solution to form a Stevia extract including steviol glycosides and/or salts thereof, and malonated steviol glycosides and/or salts thereof. The method can include converting at least some of the malonated steviol glycosides and/or salts thereof to non-malonated steviol glycosides and/or salts thereof, to produce a modified Stevia extract. The method can also include recovering the steviol glycosides and/or salts thereof from the modified Stevia extract, the recovered steviol glycosides and/or salts thereof including the non-malonated steviol glycosides and/or salts thereof. The method can be a method of increasing yield of steviol glycosides and/or salts thereof from the Stevia plant material.

### Stevia Plant Material.

The Stevia plant material can be any suitable Stevia plant material including leaves of Stevia rebaudiana. The Stevia plant material can be a dried Stevia plant material, such as having a moisture content of 0 wt % to 25 wt %, or 1 wt % to 15 wt %, or 1 wt % to 10 wt %, or less than 1 wt %, 2, 3, 4, 5, 10, 15, 20, or less than 25 wt %. The Stevia plant material can be ground, pulverized, particulated, or a combination thereof. The Stevia plant material includes steviol glycosides, salts thereof, or a combination thereof (e.g., native to the Stevia plant material). The Stevia plant material also includes one or more malonated steviol glycosides, salts thereof, or a combination thereof. A malonated steviol glycoside includes one or more malonic acid ester group. Each malonate group is esterified to a hydroxyl group on a sugar moiety (e.g., rhamnose, xylose, or another sugar) of the steviol glycoside. Each malonate group has the structure —O—C(O)—CH₂—C(O)—OH, wherein the terminal —O— corresponds to the hydroxyl group on the sugar moiety of the steviol glycoside.

### Steviol Glycoside Malonic Acid Ester.

Steviol glycoside malonic acid esters (SGMAs) or salts thereof were not previously identified in Stevia plant material. SGMAs are destroyed and/or lost during conventional processing of steviol glycoside extracts. The present inventors have discovered that by converting SGMAs to non-malonated steviol glycosides (i.e., conventional steviol glycosides) by hydrolyzing the malonic acid ester group, the total yield of steviol glycosides from Stevia plant material can be increased. In various embodiments, the present invention provides a rapid and inexpensive conversion of the SGMAs to conventional steviol glycosides to increase the total yield of conventional steviol glycosides from Stevia leaf.

Conventional Stevia leaf processing operations remove and/or destroy SGMAs. Decoloring steps, such as adding iron chloride, chemically modify the SGMAs, which are then precipitated and removed. Other decoloring steps, such as anion exchange chromatography, bind the SGMAs to the stationary phase, along with other colored molecules, while the desired traditional steviol glycosides are passed through and collected for further processing. Typical regeneration procedures for these anionic resin columns destroy the SGMAs that were bound to the resin during processing.

When the SGMA is subjected to sufficiently basic conditions, the malonic acid ester group is hydrolyzed off of the molecule, forming a conventional steviol glycoside (i.e., a non-malonated steviol glycoside). Thus, each molecule of malonated steviol glycoside that is converted is one more molecule of steviol glycoside that can be recovered and sold.

The steviol glycoside malonic acid ester (SGMA) or salt thereof. The SGMA includes one or more malonic acid ester groups, such as 1-3 malonic acid ester groups or more (e.g., no more than 1-3 malonic acid ester groups), 2 malonic acid ester groups (e.g., no more than 2 malonic acid ester groups), or 1 malonic acid ester group (e.g., no more than 1 malonic acid ester group). The malonic acid ester group can have the structure:

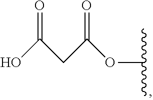 [SMILES: *OC(=O)CC(=O)O]

or a salt thereof.

The SGMA salt can be any suitable salt of the SGMA. For example, the salt can be a malonic acid salt including a counterion that is sodium, potassium, calcium, magnesium, ammonium, or a combination thereof. The salt can be a malonic acid salt including a counterion that is sodium, potassium, or a combination thereof.

The SGMA can be any suitable steviol glycoside including a malonic acid ester group. The SGMA can include one or more of glucose, xylose, rhamnose, or a combination thereof. The SGMA can have the structure:

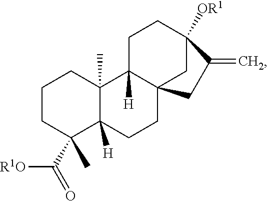

or a salt thereof. At each occurrence R can be independently chosen from —H, a malonic acid ester or a salt thereof, and a glycosidically-bonded primary sugar. At each occurrence the primary sugar can be independently chosen from glucose, xylose, and rhamnose, and at each occurrence the primary sugar can independently optionally include a secondary sugar glycosidically-bonded to the primary sugar, a malonic acid ester or a salt thereof bonded to the primary sugar, or a combination thereof. At each occurrence the secondary sugar, if present, can be independently chosen from glucose, xylose, and rhamnose, and at each occurrence the secondary sugar can independently optionally include a tertiary sugar glycosidically-bonded to the secondary sugar, a malonic acid ester or a salt thereof bonded to the secondary sugar, or a combination thereof. At each occurrence the tertiary sugar, if present, can be independently chosen from glucose, xylose, and rhamnose, and at each occurrence the tertiary sugar can independently optionally include a malonic acid ester or a salt thereof bonded to the tertiary sugar. The SGMA includes at least one of the primary sugars and at least one of the malonic acid ester groups or a salt thereof.

The SGMA can be free of the secondary sugars. The SGMA can include at least one of the secondary sugars. The SGMA can be free of the tertiary sugars. The SGMA can include at least one of the tertiary sugars. Stevia extract.

Stevia extracts are made commercially by a variety of processes. Any such Stevia extract should be suitable for treatment in accordance with aspects of the invention so long as the extract includes one or more malonated steviol glycosides, salts thereof, or a combination thereof.

If so desired, though, the method can include contacting the Stevia plant material with an aqueous extraction solution to form a Stevia extract including steviol glycosides and/or salts thereof, and malonated steviol glycosides and/or salts thereof. The Stevia extract includes one or more malonated steviol glycosides, salts thereof, or a combination thereof. The Stevia extract can include steviol glycosides, salts thereof, or a combination thereof, and one or more malonated steviol glycosides, salts thereof, or a combination thereof.

Water can be any suitable proportion of the aqueous extraction solution used to extract the Stevia plant material, such as 10 wt % to 100 wt % of the aqueous extraction solution, 30 wt % to 70 wt % of the aqueous extraction solution, or 10 wt % or more, or greater than 20 wt %, 30, 40, 50, 60, 70, 80, 85, 90, 92, 94, 95, 96, 97, 98 wt %, 99 wt % or more but less than 100%, or 100 wt %. The aqueous extraction solution can include one or more water-miscible organic solvents, such as one or more water-miscible alcohols (e.g., ethanol, methanol, or a combination thereof). For example, the aqueous extraction solution can be 50:50 water:ethanol (vol:vol). The one or more water-miscible organic solvents can form any suitable proportion of the aqueous extraction solution, such as 0 wt % to 90 wt % of the aqueous extraction solution, or 30 wt % to 70 wt % of the aqueous extraction solution, or 30 wt % or more, or less than 40 wt %, 50, 60, 70, 80, 85 wt %, or 90 wt % or less. Prior to and/or during contacting with the Stevia plant material, the aqueous extraction solution can have any suitable pH, such as a pH of 4 to 9, or 5 to 8, or 5 to 6, or 4 or more, or less than or equal to 9 but equal to or greater than 4.5, 5, 5.5, 6, 6.5, 7, 7.5, 8, or 8.5. Preferably, the pH is less than 9, more preferably less than 8.5, less than 8, or less than 7.5 because elevated pH during extraction can degrade the steviol glycosides before they are recovered.

The aqueous extraction can be performed at any suitable temperature, such as 4° C. to 100° C., 50° C. to 70° C., 15° C. to 30° C., or 4° C. or more, or less than, equal to, or greater than 6° C., 8, 10, 12, 14, 16, 18, 20, 22, 24, 26, 28, 30, 35, 40, 45, 50, 55, 60, 65, 70, 75, 80, 85, 90, 95° C., or 100° C. or less. Contacting the Stevia plant material with the aqueous extraction solution forms the Stevia extract, which includes malonated steviol glycosides and/or salts thereof. The contacting can occur for any suitable time period, such as 1 second to 300 minutes, 1 minute to 60 minutes, 10 minutes to 30 minutes, or 1 minute or more, or less than, equal to, or greater than 1 minutes, 2, 3, 4, 6, 8, 10, 12, 14, 16, 18, 20, 25, 30, 40, 50, 60, 80, 100, 120, 140, 160, 180, 200, 225, 250, 275, or 300 minutes or less. Prior to the converting of the malonated steviol glycosides and/or salts thereof to non-malonated steviol glycosides and/or salts thereof, the Stevia extract can have a pH of 4 to 9, or 5 to 8, or 5 to 6, or 4 or more, or less than or equal to 9 but equal to or greater than 4.5, 5, 5.5, 6, 6.5, 7, 7.5, 8, or 8.5. Preferably, the pH is less than 9, more preferably less than 8.5, less than 8, or less than 7.5 because elevated pH during extraction can degrade the steviol glycosides before they are recovered.

### Converting.

The method can include converting at least some of the malonated steviol glycosides and/or salts thereof in a Stevia extract to non-malonated steviol glycosides and/or salts thereof, to produce a modified Stevia extract. The converting can include holding (e.g., raising to and maintaining) the Stevia extract to at or above a suitable pH for a time and temperature sufficient to convert at least some of the malonated steviol glycosides and/or salts thereof to non-malonated steviol glycosides and/or salts thereof.

The converting can include converting any suitable amount of the malonated steviol glycosides and/or salts thereof to non-malonated steviol glycosides and/or salts thereof, such as converting 50 wt % to 100 wt % of the malonated steviol glycosides and/or salts thereof to non-malonated steviol glycosides and/or salts thereof, or 80 wt % to 100 wt %, or 50 wt % or more, or less than, equal to, or greater than 55 wt %, 60, 65, 70, 75, 80, 82, 84, 86, 88, 90, 91, 92, 93, 94, 95, 96, 97, 98, 99, 99.9, 99.99, 99.999 wt %, or 100 wt % or less. The converting can hydrolyze substantially all malonated steviol glycosides, salts thereof, or a combination thereof in the Stevia extract to non-malonated steviol glycosides, salts thereof, or a combination thereof.

The converting can include adding base to the Stevia extract to raise pH of the Stevia extract to a suitable level. The base can include any suitable one or more bases. The base can include an inorganic base. The base can include NaOH, Ca(OH)₂, KOH, or a combination thereof. The base can be added in any suitable form, such as in the form or a solid or in the form of a solution of the base (e.g., an aqueous solution of the base). The raised pH of the converting can be any suitable pH that results in hydrolysis of the malonic ester groups, such as 10 to 14, 11.1 to 14, 11.1 to 13.5, 11.5 to 13.5, 11.7 to 13.3, 12 to 13, or 10 or more, or equal to or greater than 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9. A pH of 11.1 or more can carry out the conversion at a more rapid rate, such as at a more commercially-beneficial rate. During the addition of the base to the Stevia extract, the Stevia extract can have any suitable temperature, such as 4° C. to 100° C., 50° C. to 70° C., 15° C. to 30° C., or ambient temperature, or 4° C. or more, or less than, equal to, or greater than 6° C., 8, 10, 12, 14, 16, 18, 20, 22, 24, 26, 28, 30, 35, 40, 45, 50, 55, 60, 65, 70, 75, 80, 85, 90, 95° C., or 100° C. or less.

The converting can include allowing the base to react with the Stevia extract for a suitable time and at a suitable temperature to hydrolyze a desired amount of malonated steviol glycosides, salts thereof, or a combination thereof in the Stevia extract to non-malonated steviol glycosides, salts thereof, or a combination thereof. The base can be allowed to react with the Stevia extract for 1 minute to 300 minutes, 1 minute to 60 minutes, 10 minutes to 30 minutes, or 1 minute or more, or less than, equal to, or greater than 2 minutes, 4, 6, 8, 10, 12, 14, 16, 18, 20, 25, 30, 40, 50, 60, 80, 100, 120, 140, 160, 180, 200, 225, 250, 275, or 300 minutes or less. In some aspects, the reaction of the base with the Stevia extract can be terminated by lowering the pH of the Stevia extract, such that the base is not allowed to react with the Stevia extract for longer than the upper time limit. The base can be allowed to react with the Stevia extract at a temperature of 4° C. to 100° C., 50° C. to 70° C., 15° C. to 30° C., or 4° C. or more, or ambient temperature, or less than, equal to, or greater than 6° C., 8, 10, 12, 14, 16, 18, 20, 22, 24, 26, 28, 30, 35, 40, 45, 50, 55, 60, 65, 70, 75, 80, 85, 90, 95° C., or 100° C. or less.

The specific operating parameters for commercial production can be varied as needed to achieve acceptable throughput in light of factors such as targeted production cycle time, the amount of malonated steviol glycosides in the Stevia extract, the cost of the base used to increase the pH, heating expenses, and the like. Generally, the higher the temperature and the higher the pH, the faster the rate of conversion, so the reaction times can be shorter. Thus, a substantial majority of the malonated steviol glycosides might be converted to non-malonated steviol glycosides in 15 minutes or less at a pH of 12.5 and a temperature of 30° C., but a longer reaction time might be beneficial at a pH of 10 and a temperature of 20° C.

The method can include lowering the pH of the modified Stevia extract after a suitable amount of malonated steviol glycosides and/or salts thereof are converted to non-malonated steviol glycosides and/or salts thereof. The pH of the Stevia extract can be lowered to any suitable pH, such as 4 to 9, or 5 to 8, or 5 to 6, or 4 or more, or less than 9 but equal to or greater than 4.5, 5, 5.5, 6, 6.5, 7, 7.5, 8, or 8.5. Lowering the pH of the modified Stevia extract can include adding one or more acids to the modified Stevia extract. The acid can include one or more mineral acids. The acid can include hydrochloric acid, sulfuric acid, phosphoric acid, nitric acid, acetic acid, boric acid, oxalic acid, citric acid, or a combination thereof. Phosphoric acid and hydrochloric acid are each useful in commercial production, for example. The acid can be added to the modified Stevia extract in the form of an aqueous solution including the acid. During the lowering of pH of the modified Stevia extract, the modified Stevia extract can have any suitable temperature, such as 4° C. to 100° C., 50° C. to 70° C., 15° C. to 30° C., or 4° C. or more, or ambient temperature, or less than, equal to, or greater than 6° C., 8, 10, 12, 14, 16, 18, 20, 22, 24, 26, 28, 30, 35, 40, 45, 50, 55, 60, 65, 70, 75, 80, 85, 90, 95° C., or 100° C. or less.

### Recovering and Further Processing.

The method can include recovering the steviol glycosides and/or salts thereof from the modified Stevia extract, the recovered steviol glycosides and/or salts thereof including the non-malonated steviol glycosides and/or salts thereof. The recovering can include one or more steps that increase purity of steviol glycosides and/or salts thereof in the modified Stevia extract. The recovering can include membrane filtration, ion exchange chromatography, adsorption chromatography (e.g., using an adsorption resin), column chromatography, activated carbon treatment, crystallization, treatment with FeCl₃, treatment with Ca(OH)₂, or a combination thereof. The recovering can include ion exchange chromatography.

Prior to the recovering, such as prior to lowering the pH of the modified Stevia extract, the method can be substantially free of steps that destroy or reduce the amount of malonated steviol glycosides in the extraction solution (if any is present) without transforming the malonated steviol glycosides to non-malonated steviol glycosides in the extraction solution. Prior to the recovering, the method can be substantially free of subjecting malonated steviol glycosides to ion exchange chromatography, treatment with FeCl₃, treatment with Ca(OH)₂, and activated carbon treatment.

The method can include further processing the composition including the composition including the steviol glycosides, salts thereof, or a combination thereof (e.g., before, after, or during the recovering). The further processing can include any suitable further processing, such as decolorizing, evaporating, deionizing, concentrating, drying, or a combination thereof.

The steviol glycosides, salts thereof, or combination thereof in the modified Stevia extract composition provided by the method can be provided at a yield that is 1% to 250% greater than obtained from the same method without the converting (e.g., without the adding of the base, the allowing of the base and the Stevia extract to react, and the lowering of pH), 10% to 130% greater, 20% to 83% greater, 22% to 24% greater, or 1% greater or more, or more than 5% greater, 10, 12, 14, 16, 18, 20, 21, 22, 23, 24, 25, 26, 28, 30, 35, 40, 45, 50, 55, 60, 65, 70, 75, 80, 85, 90, 95, 100, 105, 110, 115, 120, 125, 130, 140, 150, 160, 170, 180, 190, 200, 210, 220, 230, 240, or 250% greater or more. The actual increase in yield will depend in part on the content of malonated steviol glycosides and/or salts thereof relative to the steviol glycosides and/or salts thereof in the Stevia extract. A Stevia extract with more malonated steviol glycosides and/or salts thereof than steviol glycosides and/or salts thereof can achieve a steviol glycoside yield increase of 100% or more; a Stevia extract with only 10% as much malonated steviol glycosides and/or salts thereof as steviol glycosides and/or salts thereof cannot increase steviol glycoside yield by more than 10%.

The method can provide a yield or mass of major steviol glycosides from the modified extract that is at least 1% greater than a yield or mass of the major steviol glycosides (e.g., rebaudioside A, stevioside, rebaudioside C, rebaudioside D, rebaudioside F, rebaudioside M, or any combination thereof), from the Stevia extract under the same recovery conditions, such as 1% to 250%, 10% to 130% greater, 20% to 83% greater, 22% to 24% greater, or 1% greater or more, or less than, equal to, or greater than 5% greater, 10, 12, 14, 16, 18, 20, 21, 22, 23, 24, 25, 26, 28, 30, 35, 40, 45, 50, 55, 60, 65, 70, 75, 80, 85, 90, 95, 100, 105, 110, 115, 120, 125, 130, 140, 150, 160, 170, 180, 190, 200, 210, 220, 230, 240, or 250% greater or more.

The method can include forming a waste composition in addition to the composition including the steviol glycosides, salts thereof, or a combination thereof. The waste composition can have a lower concentration of malonated steviol glycosides and/or salts thereof than a waste composition formed from the same method without the converting (e.g., the adding of the base, the allowing of the base and the Stevia extract to react, and the lowering of pH). The waste composition formed by the method can have a concentration of malonated steviol glycosides and/or salts thereof of about 0 wt %.

### EXAMPLES

Various embodiments of the present invention can be better understood by reference to the following Examples which are offered by way of illustration. The present invention is not limited to the Examples given herein.

Reb A is an abbreviation for rebaudioside A. Reb B is an abbreviation for rebaudioside B.

In the extractions performed in the Examples, the Stevia leaf was ground prior to extraction. The dried leaf was ground to small particles using common grinding equipment, e.g., Retsch mill, coffee grinder, or food processor, depending on the amount of leaf processed.

In these Examples, conventional Stevia leaf extraction and post-extraction processing includes treatment with hydrophobic resin (Dowex SP70, a polyvinylstyrene-divinylbenzene cross-linked resin), cationic resin (Dowex 88) and an anionic resin (Dowex 66). After the resin purification, ethanol crystallization was used for purification.

### Example 1

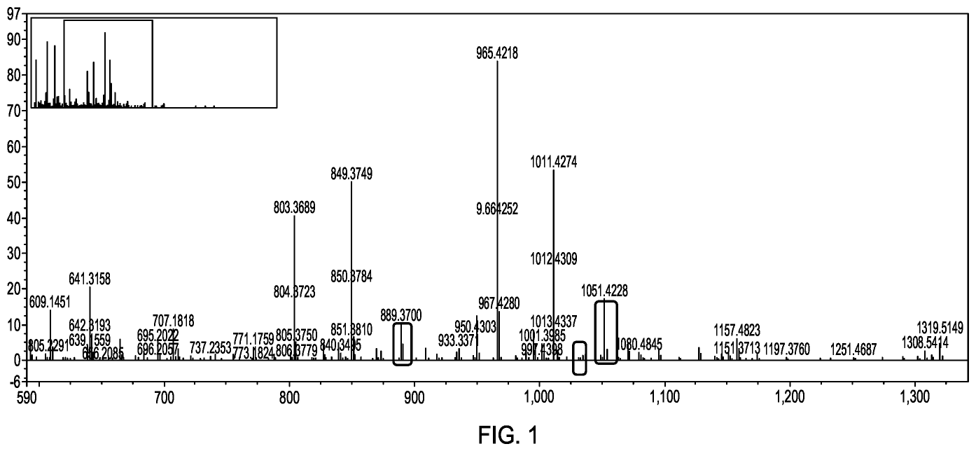

A 50% vol/vol aqueous ethanol solution was used to extract Stevia leaf (a Chinese leaf agglomerate from several lots). FIG. 1 illustrates a high-resolution mass spectrometry (HRMS) spectrum of the raw extract (diluted to 1:100), with the circled peaks corresponding to malonate-containing steviol glycosides.

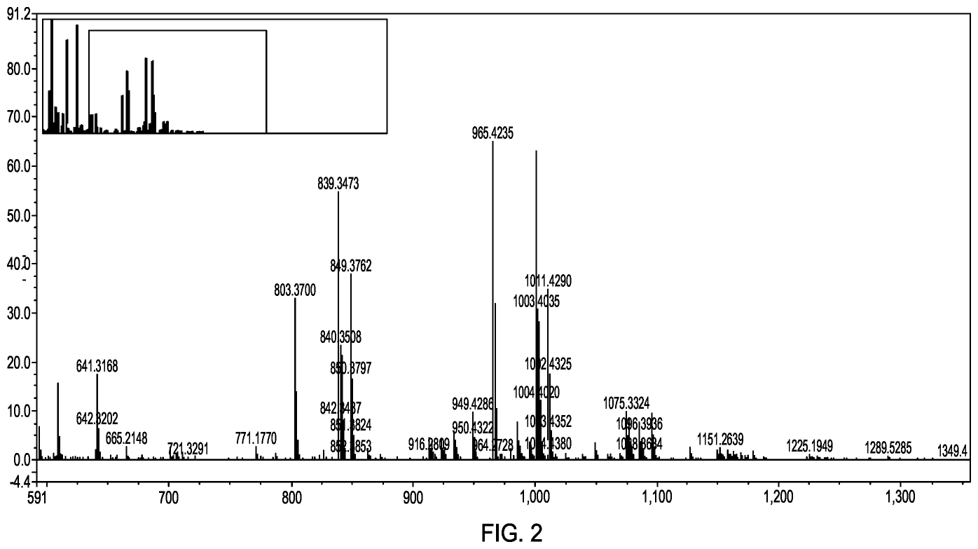

The 50% ethanol extract was subjected to hydrolysis at pH 12.5 for 20 minutes at ambient temperature (22° C.), by combining the extract with the necessary amount of an aqueous solution of 10% sodium hydroxide and stirring. FIG. 2 illustrates an HRMS spectrum of the resulting hydrolyzed raw extract (diluted to 1:100). Comparing FIGS. 1 and 2, FIG. 2 illustrates that all the malonated steviol glycosides from FIG. 1 have been converted to traditional non-malonated steviol glycosides, such as rebaudioside A (e.g., sum of peaks at m/z 965.4211, 1001.3967, and 1011.4278), stevioside (e.g., sum of peaks at m/z 803.3675, 839.3482, 849.3762), and others.

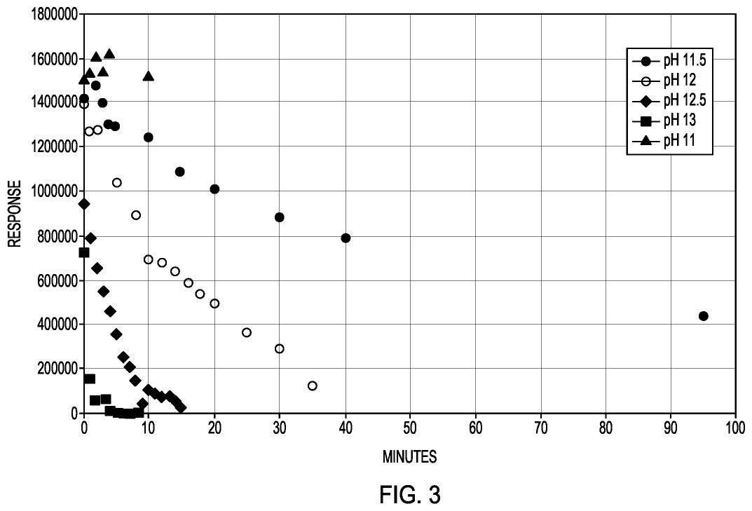

The hydrolysis of the unhydrolyzed Stevia leaf extract was repeated under various pH conditions from pH 11 to pH 13 using a similar procedure at ambient temperature (22° C.). FIG. 3 illustrates a plot of response (the integrated HRMS peak areas of the malonated SGs) versus time at each of the various pH conditions. FIG. 3 illustrates that at pH 11 and below, hydrolysis of the malonate-containing compounds does not occur or only occurs very slowly, as demonstrated by quantifying the FIA-HRMS signal arising from the exact mass of the malonated steviol glycosides at each timepoint. A pH level of 12.5 provided rapid conversion to traditional glycosides while reducing undesired degradation (e.g., degradation of Reb A to Reb B).

Ultra-high pressure liquid chromatography with a ultraviolet detector (UHPLC/UV) was used to determine the amount of steviol glycoside yield increase as a result of performing hydrolysis at pH 12.5 for 15 minutes, followed by neutralization using acid, prior to performing conventional Stevia glycoside extraction steps of decolorization, adsorbent resin chromatography, ion exchange, removal of solvent, and crystallization. Table 1 illustrates the results, comparing the concentration of glycosides in a conventional leaf extract with the concentration of glycosides in an extract having gone through the hydrolysis step, showing a yield improvement of rebaudioside A and stevioside of about 10% (i.e., [traditional steviol glycoside concentration without hydrolysis]*1.1=[traditional steviol glycoside concentration with hydrolysis]) for the particular leaf sample used (Peruvian leaf agglomerate from several lots). The amount of yield improvement will be based on the particular leaf used. The typical range of improvement can be expected to be 6-40% for most Stevia leaves, but can be 150% or more for some leaves (i.e., [traditional steviol glycoside concentration without hydrolysis]*[1.06 to 2.5 or more]=[traditional steviol glycoside concentration with hydrolysis]).

TABLE 1
Yield improvement from hydrolysis at pH 12.5 for 15 minutes
prior to acid neutralization and conventional processing.

| | % in leaf (dry wt basis) | | |
|---|---|---|---|
| | Reb A | Stevioside | Total Reb A + Stevioside |
| Stevia leaf extract | 4.27 | 1.12 | 5.39 |
| (% wt/dry wt) | | | |
| With addition of hydrolysis | 4.68 | 1.25 | 5.94 |
| (% wt/dry wt) | | | |
| % recovery | | | 110.1% |
| Mass yield increase | | | 0.55 |
| from leaf (% wt/dry wt) | | | |

The experiment was repeated, using the same conditions with a different Stevia leaf, to compare conventional post-extraction treatment to post-extraction treatment including basic hydrolysis at pH 12.5 for 15 minutes. Table 2 illustrates the results, showing a yield improvement of 42.7% from this leaf. The mass yield increase from the leaf was 3.1% (wt/dry wt).

TABLE 2
Yield improvement from hydrolysis at pH 12.5 for 15
minutes prior to acid neutralization and conventional processing.

| | Total traditional | Total malonated |
|---|---|---|
| | steviol glycosides | steviol glycosides |
| Conventionally processed leaf | 7.1 | 3.3 |
| extract | | |
| (% wt/dry wt) | | |
| With addition of hydrolysis | 10.2 | 0.1 |
| (% wt/dry wt) | | |
| % Recovery | 142.7% | 3.0% |

### Example 2

Ten Stevia leaf samples (3.0 g±0.05 g) were extracted into 30 mL water at 65° C. for 30 minutes. An aliquot of this initial extract was removed and analyzed by UHPLC-UV for steviol glycoside (SG) content. The remaining extract was adjusted to pH 12.4±0.2 using a solution of 10% wt/vol NaOH in water. The pH of the solutions was verified by pH meter and the solutions were allowed to react for 30 minutes. This process converts the malonated steviol glycosides to traditional SGs by hydrolyzing the malonic acid group. After 30 minutes, the solutions were adjusted to pH 5±1 using 4 N HCl. The resulting solutions were analyzed by UHPLC/UV for steviol glycoside content. Summary data is shown in Tables 3 and 4. SG=steviol glycoside, traditional SG=steviol glycoside with no malonic acid esters, SGMA=malonated steviol glycoside. Mass yield correlates to what total percent of the Stevia leaf was SGMAs.

TABLE 3
Glycoside profile of the ten leaf samples before hydrolysis.

| Sample | Total SGs | Total Traditional SGs | Total SGMAs |
|---|---|---|---|
| Number | % (wt/dry wt) | % (wt/dry wt) | % (wt/dry wt) |
| 1 | 10.7 | 6.69 | 3.97 |
| 2 | 9.68 | 5.73 | 3.95 |
| 3 | 14.0 | 11.4 | 2.55 |
| 4 | 13.1 | 10.5 | 2.56 |
| 5 | 8.63 | 4.66 | 3.97 |
| 6 | 12.9 | 10.9 | 1.95 |
| 7 | 13.5 | 9.33 | 4.13 |
| 8 | 12.6 | 10.1 | 2.48 |
| 9 | 13.4 | 10.5 | 2.86 |
| 10 | 6.14 | 4.78 | 1.36 |

TABLE 4
Increased yield of traditional SGs after hydrolysis.

| | Traditional SG | Traditional SG Yield | Mass Yield |
|---|---|---|---|
| | Yield | Increase | Increase from |
| Sample | (Percent | (Percentage | Leaf |
| Number | Yield) | Increase) | % (wt/dry wt) |
| 1 | 156% | 56% | +3.76 |
| 2 | 166% | 66% | +3.78 |
| 3 | 123% | 23% | +2.62 |
| 4 | 121% | 21% | +2.21 |
| 5 | 182% | 82% | +3.80 |
| 6 | 120% | 20% | +2.18 |
| 7 | 144% | 44% | +4.10 |
| 8 | 124% | 24% | +2.48 |
| 9 | 127% | 27% | +2.89 |
| 10 | 129% | 29% | +1.41 |

To illustrate the breadth of benefits of this process change, the results from Leaf Sample 3, Leaf Sample 5, and Leaf Sample 7 can be compared. Without implementing this process change, Sample 3 is the highest-yielding leaf (11.4% Total Traditional SGs) and Sample 5 is the lowest-yielding leaf (4.66% Total Traditional SGs) among this data set. However, Sample 5 has a large relative fraction of SGMA (3.97% Total SGMAs vs 4.66% Total Traditional SGs), which means that the yields are nearly doubled after the hydrolysis procedure, the highest percent increase (82%) of any sample tested here. It is still the 2^{nd}-lowest total yielding leaf, but one who only has access to very poor-quality leaf could benefit greatly from this process. On the other hand, because Sample 3 has a low relative fraction of SGMAs (2.55% Total SGMAs vs 11.4% Total Traditional SGs), the percent increase (23%) is the 3^{rd}-lowest of any sample here, despite its high total yield.

Sample 7, on the other hand, starts off being a middling producer (6^{th} best of 10 at 9.33% Total Traditional SGs), but it also has the highest starting mass of SGMAs (4.13% Total SGMAs). Properly accounting for the SGMAs, and converting them to traditional SGs by the hydrolysis procedure described here, makes Sample 7 the 2ⁿd-highest producer of total SGs (13.5% Total SGs). This illustrates that our novel hydrolysis process can change a product from being a middling producer into being an excellent source of traditional glycosides.

### Example 3

A Stevia breeding program was conducted to investigate steviol glycosides (SGs) in various plants. The plants were analyzed for presence of traditional SGs as well as malonated steviol glycosides (SGMAs).

A total of 3051 Stevia plants were grown and analyzed in this study (“Plants A”). An additional 36 plants grown by others were included in the analyses (“Plants B”). The average production of SGMAs in Plants A on a dry-weight basis was 1.7% wt/wt in the leaf, with an observed range of 0%-6% wt/wt. In Plants B, the average production was 2.6% wt/wt, with an observed range of 1%-4% wt/wt.

Among the same sets of plants, the amount of SGMAs can be compared to the amount of traditional SGs. In Plants A, the average production of SGMAs was 24% as compared to the production of traditional SGs, with a range of 0%-150% relative abundance. In Plants B, the average production of SGMAs was 22%, with a range of 6%-40% relative to traditional SGs.

Examples 1 and 2 demonstrate that treating Stevia leaf extract with base (pH approximately 12-13) can convert SGMAs to their corresponding traditional SGs in a high-yielding process with good conservation of glycoside species. By implementing this process, given the results in Examples 1-2, the average yield of the >3000 plants analyzed in this study is expected to be 24% better than provided by current Stevia processing methods, with several plants delivering >100% yield improvements (i.e., more than doubling the yield of traditional SGs).

### Example 4

Analysis of over 1,200 Stevia samples from a variety of sources showed an average of 19.6% malonated glucose-containing steviol glycosides (SGMA) relative to traditional SG content, where traditional SG content is the sum of all SGs containing either Glc, Rha, and/or Xyl sugar additions. The average SGMA/traditional SGs content was 19.6%, but some were as high as 216% (more SGMAs than traditional SGs). The amount of SGMA in common Stevia leaf was >2% on a dry weight basis.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### Exemplary Embodiments

The following exemplary embodiments are provided, the numbering of which is not to be construed as designating levels of importance:

Embodiment 1 provides a method of treating a Stevia extract that comprises steviol glycosides and/or salts thereof, and malonated steviol glycosides and/or salts thereof, the method comprising:

treating the Stevia extract at a pH greater than 10 to convert at least some of the malonated steviol glycosides and/or salts thereof to non-malonated steviol glycosides and/or salts thereof, to produce a modified Stevia extract;

decreasing the pH of the modified Stevia extract to a pH of less than 9 to provide a pH-adjusted modified Stevia extract; and

recovering the steviol glycosides and/or salts thereof from the pH-adjusted modified Stevia extract, the recovered steviol glycosides and/or salts thereof comprising the non-malonated steviol glycosides and/or salts thereof.

Embodiment 2 provides the method of Embodiment 1, wherein the method is a method of obtaining steviol glycosides from Stevia plant material, wherein the method further comprises contacting the Stevia plant material with an aqueous extraction solution (e.g., at a pH of less than 9) to form the Stevia extract.

Embodiment 3 provides the method of any one of Embodiments 1-2, wherein the converting comprises adding base to the Stevia extract to raise pH of the Stevia extract to 10 to 14, or 11.1 to 14; and

allowing the base to react with the Stevia extract for 1 minute to 300 minutes to form the modified Stevia extract; and

the method further comprises lowering the pH of the modified Stevia extract to 4 to 9.

Embodiment 4 provides the method of any one of Embodiments 1-3, comprising holding the Stevia extract at a pH of 10 to 14, or 11.1 to 14, for a time and temperature sufficient to convert 50 wt % to 100 wt % (e.g., at least 50 wt %) of the malonated steviol glycosides and/or salts thereof to non-malonated steviol glycosides and/or salts thereof.

Embodiment 5 provides the method of any one of Embodiments 1-4, comprising holding the Stevia extract at a pH of 10 to 14, or 11.1 to 14, for a time and temperature sufficient to convert 80 wt % to 100 wt % of the malonated steviol glycosides and/or salts thereof to non-malonated steviol glycosides and/or salts thereof.

Embodiment 6 provides a method of extraction of steviol glycosides from Stevia plant material, the method comprising:

contacting the Stevia plant material with an aqueous extraction solution, to form a Stevia extract;

adding base to the Stevia extract to raise pH of the Stevia extract to 10 to 14, or 11.1 to 14;

allowing the base to react with the Stevia extract for 1 minute to 300 minutes to form a modified Stevia extract;

lowering the pH of the modified Stevia extract to 4 to 9; and

recovering steviol glycosides, salts thereof, or a combination thereof from the modified Stevia extract, to form a composition comprising the steviol glycosides, salts thereof, or a combination thereof.

Embodiment 7 provides the method of any one of Embodiments 2-6, wherein the method is a method of increasing yield of steviol glycosides and/or salts thereof from the Stevia plant material.

Embodiment 8 provides the method of any one of Embodiments 2-7, wherein the Stevia plant material comprises leaves of Stevia rebaudiana.

Embodiment 9 provides the method of any one of Embodiments 2-8, wherein the Stevia plant material is a dried Stevia plant material.

Embodiment 10 provides the method of any one of Embodiments 2-9, wherein the Stevia plant material is ground, pulverized, particulated, or a combination thereof.

Embodiment 11 provides the method of any one of Embodiments 2-10, wherein the Stevia plant material comprises steviol glycosides, salts thereof, or a combination thereof.

Embodiment 12 provides the method of any one of Embodiments 2-11, wherein the Stevia plant material comprises one or more malonated steviol glycosides, salts thereof, or a combination thereof.

Embodiment 13 provides the method of any one of Embodiments 1-12, wherein the Stevia extract comprises one or more malonated steviol glycosides, salts thereof, or a combination thereof.

Embodiment 14 provides the method of any one of Embodiments 1-13, wherein the Stevia extract comprises:

steviol glycosides, salts thereof, or a combination thereof, and

one or more malonated steviol glycosides, salts thereof, or a combination thereof.

Embodiment 15 provides the method of any one of Embodiments 1-14, wherein water is 10 wt % to 100 wt % of the aqueous extraction solution.

Embodiment 16 provides the method of any one of Embodiments 1-15, wherein water is 30 wt % to 70 wt % of the aqueous extraction solution.

Embodiment 17 provides the method of any one of Embodiments 1-16, wherein water is about 100 wt % of the aqueous extraction solution.

Embodiment 18 provides the method of any one of Embodiments 1-17, wherein the aqueous extraction solution comprises one or more water-miscible organic solvents.

Embodiment 19 provides the method of Embodiment 18, wherein the one or more water-miscible organic solvents are 0 wt % to 90 wt % of the aqueous extraction solution.

Embodiment 20 provides the method of any one of Embodiments 18-19, wherein the one or more water-miscible organic solvents are 30 wt % to 70 wt % of the aqueous extraction solution.

Embodiment 21 provides the method of any one of Embodiments 18-20, wherein the one or more water-miscible organic solvents comprise a water-miscible alcohol.

Embodiment 22 provides the method of any one of Embodiments 18-21, wherein the one or more water-miscible organic solvents comprise ethanol, methanol, or a combination thereof.

Embodiment 23 provides the method of any one of Embodiments 1-22, wherein the aqueous extraction solution comprises water and also comprises ethanol, methanol, or a combination thereof.

Embodiment 24 provides the method of any one of Embodiments 1-23, wherein prior to the converting, the Stevia extract has a pH of 4 to 9.

Embodiment 25 provides the method of any one of Embodiments 1-24, wherein prior to the converting, the Stevia extract has a pH of 5 to 6.

Embodiment 26 provides the method of any one of Embodiments 1-25, wherein the extraction is performed at a temperature of 4° C. to 100° C.

Embodiment 27 provides the method of any one of Embodiments 1-26, wherein the extraction is performed at a temperature of 50° C. to 70° C.

Embodiment 28 provides the method of any one of Embodiments 1-27, wherein the extraction is performed at a temperature of 15° C. to 30° C.

Embodiment 29 provides the method of any one of Embodiments 3-28, wherein the modified Stevia extract comprising the base has a pH of 11.5 to 13.5.

Embodiment 30 provides the method of any one of Embodiments 3-29, wherein the modified Stevia extract comprising the base has a pH of 12 to 13.

Embodiment 31 provides the method of any one of Embodiments 3-30, wherein the base is an inorganic base.

Embodiment 32 provides the method of any one of Embodiments 3-31, wherein the base comprises NaOH, Ca(OH)₂, KOH, or a combination thereof.

Embodiment 33 provides the method of any one of Embodiments 3-32, wherein the base is added to the Stevia extract in the form of an aqueous solution comprising the base.

Embodiment 34 provides the method of any one of Embodiments 1-33, wherein the Stevia extract has a temperature of 4° C. to 100° C. during the addition of the base thereto.

Embodiment 35 provides the method of any one of Embodiments 1-34, wherein the Stevia extract has a temperature of 15° C. to 30° C. during the addition of the base thereto.

Embodiment 36 provides the method of any one of Embodiments 3-35, wherein the base is allowed to react with the Stevia extract for 1 minute to 60 minutes.

Embodiment 37 provides the method of any one of Embodiments 3-36, wherein the base is allowed to react with the Stevia extract for 10 minutes to 30 minutes.

Embodiment 38 provides the method of any one of Embodiments 3-37, wherein the base is allowed to react with the Stevia extract at a temperature of 4° C. to 100° C.

Embodiment 39 provides the method of any one of Embodiments 3-38, wherein the base is allowed to react with the Stevia extract at a temperature of 15° C. to 30° C.

Embodiment 40 provides the method of any one of Embodiments 3-39, wherein the base is allowed to react with the Stevia extract at ambient temperature.

Embodiment 41 provides the method of any one of Embodiments 1-40, wherein the converting is sufficient to hydrolyze substantially all malonated steviol glycosides, salts thereof, or a combination thereof in the Stevia extract to non-malonated steviol glycosides, salts thereof, or a combination thereof.

Embodiment 42 provides the method of any one of Embodiments 3-41, wherein the base is allowed to react with the Stevia extract for a time and at a temperature sufficient to hydrolyze substantially all malonated steviol glycosides, salts thereof, or a combination thereof in the Stevia extract to non-malonated steviol glycosides, salts thereof, or a combination thereof.

Embodiment 43 provides the method of any one of Embodiments 3-42, wherein the modified Stevia extract having the lowered pH has a pH of 4 to 9.

Embodiment 44 provides the method of any one of Embodiments 3-43, wherein the modified Stevia extract having the lowered pH has a pH of 5 to 7.

Embodiment 45 provides the method of any one of Embodiments 3-44, wherein the lowering of the pH of the modified Stevia extract comprises adding acid to the modified Stevia extract.

Embodiment 46 provides the method of Embodiment 45, wherein the acid comprises one or more mineral acids.

Embodiment 47 provides the method of any one of Embodiments 45-46, wherein the acid comprises hydrochloric acid, sulfuric acid, phosphoric acid, nitric acid, acetic acid, boric acid, oxalic acid, citric acid, or a combination thereof.

Embodiment 48 provides the method of any one of Embodiments 45-47, wherein the acid comprises phosphoric acid.

Embodiment 49 provides the method of any one of Embodiments 45-48, wherein the acid comprises HCl.

Embodiment 50 provides the method of any one of Embodiments 45-49, wherein the acid is added to the modified Stevia extract in the form of an aqueous solution comprising the acid.

Embodiment 51 provides the method of any one of Embodiments 3-50, wherein the modified Stevia extract has a temperature of 4° C. to 100° C. during the lowering of the pH thereof.

Embodiment 52 provides the method of any one of Embodiments 3-51, wherein the modified Stevia extract has a temperature of 15° C. to 30° C. during the lowering of the pH thereof.

Embodiment 53 provides the method of any one of Embodiments 1-52, wherein the recovering comprises one or more steps that increase purity of steviol glycosides and/or salts thereof in the modified Stevia extract.

Embodiment 54 provides the method of any one of Embodiments 1-53, wherein the recovering comprises membrane filtration, ion exchange chromatography, adsorption chromatography, column chromatography, activated carbon treatment, crystallization, treatment with FeCl₃, treatment with Ca(OH)₂, or a combination thereof.

Embodiment 55 provides the method of any one of Embodiments 1-54, wherein the recovering comprises ion exchange chromatography.

Embodiment 56 provides the method of any one of Embodiments 1-55, wherein prior to lowering the pH of the modified Stevia extract, the method is substantially free of subjecting the Stevia extract and the modified Stevia extract to ion exchange chromatography, treatment with FeCl₃, treatment with Ca(OH)₂, and activated carbon treatment.

Embodiment 57 provides the method of any one of Embodiments 1-56, further comprising further processing the composition comprising the steviol glycosides, salts thereof, or a combination thereof.

Embodiment 58 provides the method of Embodiment 57, wherein the further processing comprises decolorizing, evaporating, deionizing, concentrating, drying, or a combination thereof.

Embodiment 59 provides the method of any one of Embodiments 1-58, wherein the steviol glycosides, salts thereof, or combination thereof, in the composition are obtained at a yield that is 1% to 250% greater than obtained from the same method without the converting, or without the adding of the base, the allowing of the base and the Stevia extract to react, and the lowering of pH.

Embodiment 60 provides the method of any one of Embodiments 1-59, wherein the steviol glycosides, salts thereof, or combination thereof, in the composition are obtained at a yield that is 10% to 130% greater than obtained from the same method without the converting, or without the adding of the base, the allowing of the base and the Stevia extract to react, and the lowering of pH.

Embodiment 61 provides the method of any one of Embodiments 1-60, wherein the steviol glycosides, salts thereof, or combination thereof, in the composition are obtained at a yield from the Stevia plant material that is 20% to 83% greater than obtained from the same method without the converting, or without the adding of the base, the allowing of the base and the Stevia extract to react, and the lowering of pH.

Embodiment 62 provides the method of any one of Embodiments 1-61, wherein the steviol glycosides, salts thereof, or combination thereof, in the composition are obtained at a yield from the Stevia plant material that is 22% to 24% greater than obtained from the same method without the converting, or without the adding of the base, the allowing of the base and the Stevia extract to react, and the lowering of pH.

Embodiment 63 provides the method of any one of Embodiments 1-62, wherein a yield of major steviol glycosides recovered from the modified extract is at least 25% greater than a yield of the major steviol glycosides from the Stevia extract under the same recovery conditions.

Embodiment 64 provides the method of any one of Embodiments 1-63, wherein the method further comprises forming a waste composition in addition to the composition comprising the steviol glycosides, salts thereof, or a combination thereof, wherein the waste composition has a lower concentration of malonated steviol glycosides and/or salts thereof than a waste composition formed from the same method without the adding of the base, the allowing of the base and the Stevia extract to react, and the lowering of pH.

Embodiment 65 provides the method of Embodiment 64, wherein the waste composition formed by the method has a concentration of malonated steviol glycosides and/or salts thereof of about 0 wt %.

Embodiment 66 provides a method of increasing yield of steviol glycosides and/or salts thereof from a Stevia plant material, the method comprising:

contacting the Stevia plant material with an aqueous extraction solution having a pH of 4 to 9, to form a Stevia extract;

adding base to the Stevia extract to raise pH of the Stevia extract to 12 to 13;

allowing the base to react with the Stevia extract at 15° C. to 30° C. for 1 minute to 60 minutes to form a modified Stevia extract;

lowering the pH of the modified Stevia extract to 5 to 7; and

recovering steviol glycosides, salts thereof, or a combination thereof from the modified Stevia extract, to form a composition comprising the steviol glycosides, salts thereof, or a combination thereof;

wherein the steviol glycosides, salts thereof, or combination thereof, in the composition are obtained at a yield that is 1% to 250% greater than obtained from the same method without the adding of the base, the allowing of the base and the Stevia extract to react, and the lowering of pH.

Embodiment 67 provides the method of any one or any combination of Embodiments 1-66 optionally configured such that all elements or options recited are available to use or select from.

1. A method of treating a Stevia extract that comprises steviol glycosides and/or salts thereof, and malonated steviol glycosides and/or salts thereof, the method comprising:
treating the Stevia extract at a pH greater than 10 to convert at least some of the malonated steviol glycosides and/or salts thereof to non-malonated steviol glycosides and/or salts thereof, to produce a modified Stevia extract;
decreasing the pH of the modified Stevia extract to a pH of less than 9 to provide a pH-adjusted modified Stevia extract; and
recovering the steviol glycosides and/or salts thereof from the pH-adjusted modified Stevia extract, the recovered steviol glycosides and/or salts thereof comprising the non-malonated steviol glycosides and/or salts thereof.

2. The method of claim 1, wherein the method is a method of obtaining steviol glycosides from Stevia plant material, wherein the method further comprises contacting the Stevia plant material with an aqueous extraction solution to form the Stevia extract.

3. The method of claim 1, wherein the treating converts at least 50 wt % of the malonated steviol glycosides and/or salts thereof to non-malonated steviol glycosides and/or salts thereof.

4. The method of claim 1, wherein the converting comprises
adding base to the Stevia extract to raise pH of the Stevia extract to 10 to 14; and
allowing the base to react with the Stevia extract for 1 minute to 300 minutes to form the modified Stevia extract; and
the method further comprises lowering the pH of the modified Stevia extract to 4 to 9.

5. The method of claim 1, wherein the method is a method of increasing yield of steviol glycosides and/or salts thereof from the Stevia plant material.

6. The method of claim 1, wherein prior to the converting, the Stevia extract has a pH of 4 to 9.

7. The method of claim 1, wherein the modified Stevia extract has a pH of 11.1 to 14.

8. The method of claim 1, wherein the modified Stevia extract has a pH of 12 to 13.

9. The method of claim 4, wherein the base is allowed to react with the Stevia extract for 10 minutes to 30 minutes.

10. The method of claim 4, wherein the base is allowed to react with the Stevia extract at a temperature of 4° C. to 100° C.

11. The method of claim 4, wherein the base is allowed to react with the Stevia extract at ambient temperature.

12. The method of claim 1, wherein the converting is sufficient to hydrolyze substantially all malonated steviol glycosides, salts thereof, or a combination thereof in the Stevia extract to non-malonated steviol glycosides, salts thereof, or a combination thereof.

13. The method of claim 1, wherein the pH-adjusted modified Stevia extract has a pH of 4 to 9.

14. The method of claim 1, wherein the decreasing of the pH of the modified Stevia extract comprises adding one or more acids to the modified Stevia extract.

15. The method of claim 1, wherein the recovering comprises membrane filtration, ion exchange chromatography, adsorption chromatography, column chromatography, activated carbon treatment, crystallization, treatment with FeCl₃, treatment with Ca(OH)₂, or a combination thereof.

16. The method of claim 1, further comprising further processing the composition comprising the steviol glycosides, salts thereof, or a combination thereof, the further processing comprising decolorizing, evaporating, deionizing, concentrating, drying, or a combination thereof.

17. The method of claim 1, wherein the steviol glycosides, salts thereof, or combination thereof, in the composition are obtained at a yield that is 1% to 250% greater than obtained from the same method without the converting, or without the adding of the base, the allowing of the base and the Stevia extract to react, and the lowering of pH.

18. The method of claim 1, wherein a yield of major steviol glycosides recovered from the modified extract is at least 25% greater than a yield of the major steviol glycosides from the Stevia extract under the same recovery conditions.

19. The method of claim 1, wherein the method further comprises forming a waste composition in addition to the composition comprising the steviol glycosides, salts thereof, or a combination thereof, wherein the waste composition has a lower concentration of malonated steviol glycosides and/or salts thereof than a waste composition formed from the same method without the adding of the base, the allowing of the base and the Stevia extract to react, and the lowering of pH.

20. A method of increasing yield of steviol glycosides and/or salts thereof from a Stevia plant material, the method comprising:
contacting the Stevia plant material with an aqueous extraction solution having a pH of 4 to 9, to form a Stevia extract;
adding base to the Stevia extract to raise pH of the Stevia extract to 11.1 to 14;
allowing the base to react with the Stevia extract at 15° C. to 30° C. for 1 minute to 60 minutes to form a modified Stevia extract;
lowering the pH of the modified Stevia extract to 5 to 7; and
recovering steviol glycosides, salts thereof, or a combination thereof from the modified Stevia extract, to form a composition comprising the steviol glycosides, salts thereof, or a combination thereof,
wherein the steviol glycosides, salts thereof, or combination thereof, in the composition are obtained at a yield that is 1% to 250% greater than obtained from the same method without the adding of the base, the allowing of the base and the Stevia extract to react, and the lowering of pH.
